(19) United States
(12) Patent Application Publication
(10) Pub. No.: US 2025/0177746 A1
(43) Pub. Date: Jun. 5, 2025

(54) NON-INVASIVE STIMULATION DEVICE AND METHOD

(71) Applicant: NEUROEN LTD., Wonju-si, Gangwon-do (KR)

(72) Inventors: Jae Won LEE, Seocho-gu Seoul (KR); Do Yeun OH, Wonju-si Gangwon-do (KR)

(73) Assignee: NEUROEN LTD., Wonju-si, Gangwon-do (KR)

(21) Appl. No.: 18/842,065

(22) PCT Filed: Feb. 24, 2023
(86) PCT No.: PCT/KR2023/002662

§ 371 Date: Aug. 28, 2024

(30) Foreign Application Priority Data
Feb. 28, 2022 (CN) 10-2022-0025721

(51) Int. Cl. A61N 1/36; A61N 1/04; A61N 1/20

(52) U.S. Cl. CPC A61N 1/36031; A61N 1/0476; A61N 1/0492; A61N 1/20; A61N 1/36025; A61N 1/36034

(57) ABSTRACT

The present invention relates to a non-invasive stimulation device and method, the device comprising: a helmet in which a plurality of electrodes each including a patch configured to come into close contact with the head of a subject are disposed; at least one sensor module for sensing biometric information of a subject wearing the helmet; and a control unit for applying a direct current stimulation signal to at least one selected electrode in a transcranial direct current stimulation (tDCS) step, providing an alternating current stimulation signal to at least one selected electrode in a transcranial alternating current stimulation (tACS) step, and automatically switching from the tDCS step to the tACS step when an abnormal state in which a resistance value of the electrode exceeds a reference value or the biometric information exceeds a normal range is detected.

### TECHNICAL FIELD

The invention relates to a non-invasive stimulation device and method suitable for a brain stimulation healthcare system.

### BACKGROUND ART

The number of patients receiving treatment for mental and behavioral disorders is increasing by more than 10% annually, but a level of quality of treatment for these disorders is significantly low. In comparison, the total number of patients receiving treatment for the disease has not changed much. The social and economic burden due to mental and behavioral disorders is rapidly increasing.

Electromagnetic technology using brain stimulation therapy is gaining attention for the treatment of mental and behavioral disorders. Brain stimulation therapy includes invasive treatments such as deep brain stimulation and non-invasive treatments that stimulate the brain from the outside using magnetism, electricity, or ultrasound. However, invasive treatments always have the risk of side effects or complications after surgery, so research on non-invasive treatments is actively being conducted.

Non-invasive treatment of mild cognitive impairment does not show immediate effect compared to invasive treatment, but has the advantage of fewer side effects and a significant effect compared to cognitive treatment.

A representative example of non-invasive treatment is a method using transcranial current stimulation (tCS), and specifically, a method of activating a specific part of the subject's brain or causing rest with transcranial direct current stimulation (tDCS) is widely used.

The treatment using tDCS is less effective when the subject's tension or stress is high. Conventionally, in order to increase the effect of tDCS, an attempt has been made to lower the subject's tension or stress by playing music, etc., but there is a problem that the effect varies greatly from person to person.

tDCS causes discomfort such as a stinging pain in the skin at the beginning of the procedure, and this discomfort reduces the likelihood that the subject will use the non-invasive stimulation device regularly and in the long term. Even if it is used, there is a problem that it increases the subject's tension or stress.

If the adhesion of the patch that adheres to the electrical stimulation area during tDCS treatment decreases, or if the subject's tension or stress increases, the treatment effect will decrease. In this case, the electrical stimulation can be temporarily stopped, but it can take a considerable amount of time before the treatment is resumed.

In addition, in order to increase the effectiveness of non-invasive treatment, periodic and continuous treatment is required. However, it is practically impossible for the subject to receive treatment more than once or twice a day at a hospital. In addition, the method of using electrical stimulation, which is a representative example of non-invasive treatment of mild cognitive impairment, is to apply electrical stimulation by attaching electrodes to the scalp. Conventional electrical stimulation devices available for mild cognitive impairment have a problem that it is too difficult for ordinary people to wear. For example, there are problems in that it is difficult to position the plurality of electrodes of the electrical stimulation device in an appropriate position, and the adhesion of the electrodes is prevented by hair.

As a related prior literature, Korean Patent Application Publication No. 10-2014-0080299 (Jun. 30, 2014) is published.

### DISCLOSURE

### Technical Problem

The present invention has been made to solve the aforementioned needs and/or problems, and provides a non-invasive stimulation device and method for enhancing brain stimulation effects.

Meanwhile, other unspecified purposes of the present invention will be additionally considered within the scope that can be easily inferred from the following detailed description and its effects.

### Technical Solution

A non-invasive stimulation device according to an embodiment of the present invention comprises a helmet in which a plurality of electrodes each including a patch configured to adhere to a head of a subject are disposed; at least one sensor module configured to sense biometric information of the subject wearing the helmet; and a control unit configured to apply a direct current stimulation signal to at least one selected electrode in a transcranial direct current stimulation (tDCS) step, provide an alternating current stimulation signal to at least one selected electrode in a transcranial alternating current stimulation (tACS) step, and automatically switch from the tDCS step to the tACS step when an abnormal state in which a resistance value of the electrode exceeds a reference value or the biometric information exceeds a normal range is detected.

The control unit may apply the alternating current signal that swings around a base level in a first tACS step to the electrode selected in the tDCS step, increase an intensity of a current applied to the electrode selected in the tACS step to an activation level higher than the base level during a ramp-up period after the first tACS step, enter a second tACS step and apply the alternating current signal that swings around the activation level to the electrode selected in the tACS step when the abnormal state is detected, and resume the tDCS step after a predetermined period of time when a normal state in which the resistance value of the electrode is lower than or equal to the reference value and the biometric information changes within a normal range is detected, and then gradually lowers the intensity of the current applied to the electrode selected in the tACS step from the activation level to the base level during a ramp-down period.

The control unit may apply a current of the base level to the electrodes for a predetermined standby time immediately after a power of the non-invasive stimulation device is turned on, apply a patch detection signal pattern in a form of the alternating current signal to the electrodes when the start key input is received, measures resistances of the electrodes, compares the resistance value of each of the electrodes with the reference value, and determines a degree of adhesion of the patch, and enter the first tACS step after the patch detection signal pattern.

An amplitude of the alternating current signal generated in the first tACS step may be greater than an amplitude of the patch detection signal pattern.

An amplitude of the alternating current signal generated in the second tACS step may be lower than an amplitude of the alternating current signal generated in the first tACS step.

A frequency of the alternating current signal generated in the second tACS step may be different from a frequency of the alternating current signal generated in the first tACS step.

The alternating current signal generated in at least one of the first tACS step and the second tACS step may include a section in which an amplitude changes.

A non-invasive stimulation method according to an embodiment of the present invention may comprise the steps of measuring a resistance value of the electrodes and comparing the resistance value with a reference value; receiving an output signal of the sensor module and comparing the biometric information with a normal range; and automatically switching from the tDCS step to the tACS step when an abnormal state in which the resistance value exceeds the reference value or the biometric information exceeds the normal range is detected.

The non-invasive stimulation method may further comprise the steps of applying the alternating current signal that swings around a base level in a first tACS step to the electrode selected in the tDCS step; increasing an intensity of a current applied to the electrode selected in the tACS step to an activation level higher than the base level during a ramp-up period after the first tACS step; entering a second tACS step and applying the alternating current signal that swings around the activation level to the electrode selected in the tACS step when the abnormal state is detected; resuming the tDCS step after a predetermined period of time when a normal state in which the resistance value of the electrode is lower than or equal to the reference value and the biometric information changes within a normal range is detected; and gradually lowering the intensity of the current applied to the electrode selected in the tACS step from the activation level to the base level during a ramp-down period after the tDCS step is resumed.

The non-invasive stimulation method may further comprise the steps of applying a current of the base level to the electrodes for a predetermined standby time immediately after a power of the non-invasive stimulation device is turned on, applying a patch detection signal pattern in a form of the alternating current signal to the electrodes, measuring resistances of the electrodes, comparing the resistance value of each of the electrodes with the reference value, and determining a degree of adhesion of the patch; and entering the first tACS step after the patch detection signal pattern.

### Advantageous Effects

According to the present invention, since mild cognitive impairment can be prevented by being mounted on equipment such as a helmet, the subject can safely stimulate the brain.

The present invention can be manipulated to operate according to a set program, so that the subject can perform brain stimulation safely and conveniently.

The present invention is in the form of a helmet worn on the head of the subject, so that the subject can easily wear it by himself or herself.

The non-invasive stimulation device and method of the present invention can perform tACS stimulation on the subject when an unstable state such as a low patch adhesion, a device malfunction, or the subject's physical instability is detected during the tDCS treatment period, thereby performing tDCS electrical stimulation in a stabilized state of the device and subject, thereby enhancing the brain stimulation effect.

Furthermore, the non-invasive stimulation device and method of the present invention can provide the advantages of the tACS stimulation effect, such as the effect of synchronizing electrical stimulation and brain waves (neuro entrainment), while maintaining the tDCS stimulation effect by performing tACS stimulation during the tDCS treatment period.

The effects of the present invention are not limited to the effects mentioned above, and other effects not mentioned will be clearly understood by those skilled in the art from the description of the claims.

### DESCRIPTION OF DRAWINGS

FIG. 1 is a block diagram schematically illustrating a non-invasive stimulation device according to an embodiment of the present invention.

FIG. 2 is a diagram illustrating a brain stimulation site of a subject where the patches of the electrode modules illustrated in FIG. 1 are in contact.

FIG. 3 is a waveform diagram schematically showing a tDCS waveform and tACS waveform in an electrical stimulation signal.

FIGS. 4A and 4B are diagrams illustrating a helmet in which a non-invasive stimulation device according to an embodiment of the present invention is implemented from various angles.

FIG. 5 is an exploded perspective view of an electrode module excluding a patch.

FIG. 6 is a partial cutaway perspective view illustrating an electrode module with a contact pad mounted in a non-conductive silicone holder.

FIG. 7 is a flowchart showing a control method of a non-invasive stimulation method according to an embodiment of the present invention in time series.

FIG. 8 is a waveform diagram showing an electrical stimulation signal according to an embodiment of the present invention.

FIGS. 9A to 9C are waveform diagrams showing waveforms of electrical stimulation signals according to other embodiments of the present invention.

FIGS. 10A to 10C are diagrams illustrating an example of electrode polarity in tACS brain stimulation.

FIG. 11 is a diagram illustrating an example of electrode polarity change when switching from tDCS brain stimulation to tACS brain stimulation.

It should be understood that the accompanying drawings are exemplified by reference for understanding the technical idea of the present invention, and the scope of the present invention is not limited thereby.

### BEST MODE FOR INVENTION

A non-invasive stimulation device according to an embodiment of the present invention is provided with a helmet in which a plurality of electrodes each including a patch configured to adhere to a head of a subject are disposed; at least one sensor module configured to sense biometric information of the subject wearing the helmet; and a control unit configured to apply a direct current stimulation signal to at least one selected electrode in a transcranial direct current stimulation (tDCS) step, provide an alternating current stimulation signal to at least one selected electrode in a transcranial alternating current stimulation (tACS) step, and automatically switch from the tDCS step to the tACS step when an abnormal state in which a resistance value of the electrode exceeds a reference value or the biometric information exceeds a normal range is detected.

### MODE FOR INVENTION

Hereinafter, the configuration of the present invention guided by various embodiments of the present invention and effects resulting from the configuration will be described with reference to the drawings. In the description of the present invention, if it is determined that related known functions are obvious to those skilled in the art and can unnecessarily obscure the gist of the present invention, the detailed description thereof will be omitted.

As used herein, the term “module” can include a unit implemented in hardware, software, or firmware, and can be used interchangeably with terms such as, for example, logic, logic block, component, or circuit. A module can be an integrally formed component or a minimum unit or a part of the component that performs one or more functions.

In this disclosure, a “module” or “node” performs operations such as moving, storing, and converting data by using an arithmetic device such as a CPU or AP. For example, a “module” or “node” can be implemented as a device such as a server, PC, tablet PC, smartphone, and the like.

Hereinafter, a non-invasive stimulation device according to an embodiment of the present invention will be described with reference to the drawings.

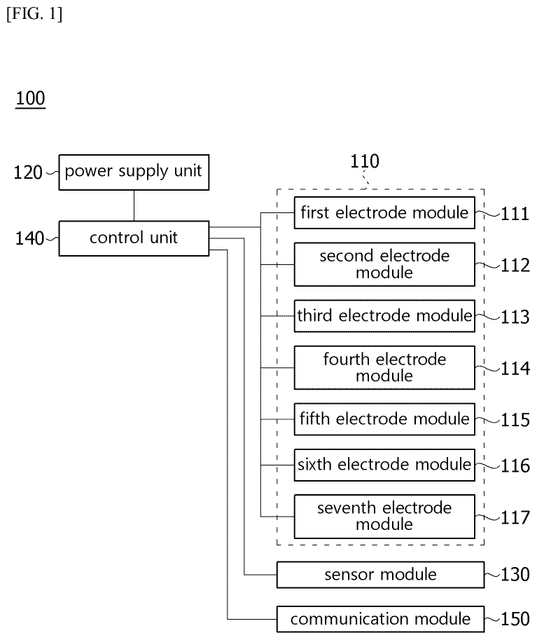

FIG. 1 is a block diagram schematically illustrating a non-invasive stimulation device according to an embodiment of the present invention.

A non-invasive stimulation device 100 according to an embodiment of the present invention is configured to non-invasively apply electrical stimulation through a patch that adheres to the scalp of a subject. Various brain-related diseases can be prevented, treated, and managed through such electrical stimulation. For example, the non-invasive stimulation device 100 of the present invention can be used to prevent, treat, and manage not only mild cognitive impairment but also insomnia, depression, convulsive disease, pain, Further it can be used for memory enhancement and motor learning ability enhancement, intellectual disability, addiction disease, and schizophrenia.

The non-invasive stimulation device 100 according to an embodiment of the present invention includes a stimulation device 110, a power supply unit 120, and a control unit 140.

The stimulation device 110 includes electrode modules 111 to 117 that apply current to a plurality of patches that adhere to various locations on the head of a subject. In the embodiment, the electrode modules 111 to 117 of the stimulation device 110 are exemplified as seven, but are not limited thereto. For example, the stimulation device 110 can include three or more electrode modules.

The control unit 140 supplies power from the power supply unit 120 and causes current to flow through the electrode modules 111 to 117. Some of the plurality of electrode modules 111 to 117 can become positive electrodes, others can become negative electrodes, and the polarity of the current applied to each of the electrode modules 111 to 117 can be reversed. In addition, it is also possible that current flows only to some of the plurality of electrode modules 111 to 117 and no current flows to other electrode modules. That is, the polarity or operation of each electrode part can be changed according to a program set in the control unit 140.

When five electrode modules are arranged in the non-invasive stimulation device 100 without the third and fourth electrode modules, the first electrode module 111 can be adhered to a position corresponding to the left frontal lobe of the subject, the second electrode module 112 can be adhered to a position corresponding to the right frontal lobe of the subject, the fifth electrode module 115 can be adhered to a position corresponding to an area including at least a portion of the left parietal lobe, left occipital lobe, and left temporal lobe of the subject, and the sixth electrode module 116 can be adhered to a position corresponding to an area including at least a portion of the right parietal lobe, right occipital lobe, and right temporal lobe of the subject. In addition, the seventh electrode module 117 can be adhered to the head of the subject at a different position from the positions of the electrode modules 111, 112, 115, and 116, and for example, can be adhered to an area including at least a portion of the back of the ear of the subject, the back of the head of the subject, and back of the neck of the subject. The seventh electrode module 117 can be a positive electrode from which current is output or negative electrode from which current is input.

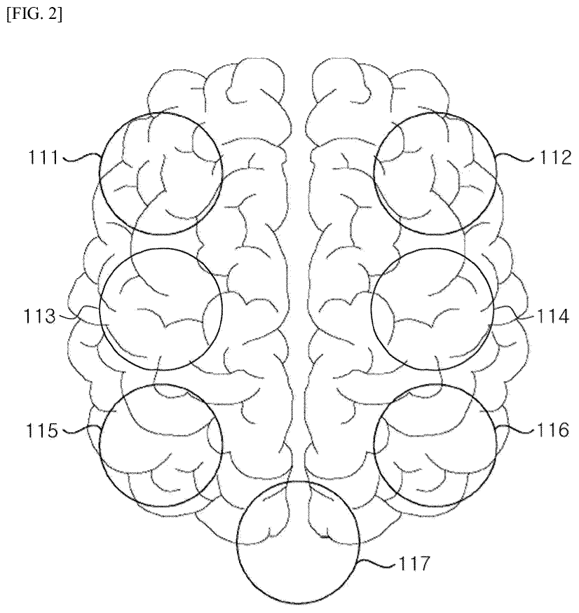

When seven electrode modules are arranged in the non-invasive stimulation device 100, as illustrated in FIG. 2, the first electrode module 111 can be adhered to a position corresponding to the left frontal lobe of the subject, the second electrode module 112 can be adhered to a position corresponding to the right frontal lobe of the subject, the third electrode module 113 can be adhered to a left motor sensory area between the left frontal lobe and left parietal lobe of the subject, and the fourth electrode module 114 can be adhered to a position corresponding to the right motor sensory area between the right frontal lobe and right parietal lobe of the subject. The fifth electrode module 115 can be adhered to a left temporoparietal lobe area of the subject, and the sixth electrode module 116 can be adhered to a right temporoparietal lobe area of the subject. In addition, the seventh electrode module 117 can be adhered to an area including at least a portion of the back of the ear of the subject, back of the head of the subject, and back of the neck of the subject. The seventh electrode module 117 can be a positive electrode from which current is output or negative electrode from which current is input.

Each of the electrode modules 111 to 117 is combined with a patch adhered to the head of the subject and applies current to the patch to apply electrical stimulation to the brain of the subject. Each of the patches can be made of a porous material, such as a sponge, that is easily compressed and restored, and can include a wet pad or dry pad containing moisture. The dry pad can be made of a multilayer hydrogel composite.

The control unit 140 can apply electrical stimulation signals to the electrode modules 111 to 117 according to a preset program according to the symptoms of the subject so that transcranial current stimulation (tCS) is applied to a desired area of the subject's head. The type of transcranial current stimulation used in the present invention can be at least one of transcranial direct current stimulation (tDCS), transcranial alternating current stimulation (tACS), and transcranial random-noise stimulation (tRNS), or a combination thereof.

The non-invasive stimulation device 100 of the present invention can be implemented in a helmet form that is easy for the subject to wear on his or her head. When the subject wears the non-invasive stimulation device 100 and starts brain stimulation treatment, the control unit 140 can apply an electric stimulation signal as illustrated in FIG. 2.

The power supply unit 120 can supply power required for the operation of the non-invasive stimulation device 100 through the power supply. The power supply unit 120 can supply power required for driving the electrode modules 111 to 117 and can be a battery if necessary. If the power supply unit 120 is a battery, a rechargeable secondary battery can be used.

The memory of the control unit 140 stores a plurality of stimulation signal patterns for providing an electric stimulation signal optimized for the subject's symptoms. The control unit 140 can select an electric stimulation position and an electric stimulation signal pattern according to the patient's symptoms or diagnosis results.

The control unit 140 can control the current amount of the electric stimulation signal supplied to each of the electrode modules 111 to 117 by controlling the power supplied from the power supply unit 120.

The non-invasive stimulation device 100 of the present invention can further include one or more sensor modules 130 for measuring various conditions of the user. The sensor module 130 can be adhered to at least one area of the head of the subject, for example, the back of the ear of the subject, the back of the head of the subject, and the back of the neck of the subject. The sensor module 130 can measure more effective biometric information, such as oxygen saturation, heart rate, stress index, and brain waves, and provide the same to the control unit 140.

The control unit 140 can analyze the biometric information received from the sensor module 130 to measure the physical changes of the subject in real time and monitor the physical changes of the subject during the electric stimulation treatment of the brain of the subject. The control unit 140 can analyze the changes in the body of the subject during the electrical stimulation treatment and automatically change the pattern or current intensity of the electrical stimulation signal applied to the electrode modules 111 to 117.

The control unit 140 can measure the voltage of each of the electrode modules 111 to 117 to which the current of the electrical stimulation signal is applied to determine the patch resistance of the electrode modules 111 to 117. When the patch is adhered to the head of the subject, the patch resistance value is lower than a preset reference value, but when the patch is not adhered to the head of the subject, the patch resistance value is measured higher than the reference value. Therefore, the control unit 140 can detect the degree of adherence of each of the patches that come into contact with the head of the subject in real time.

The control unit 140 can adjust the current intensity flowing to each of the electrode modules 111 to 117 according to the selection of the user, for example, the medical staff or the subject himself, in response to an input signal received through a user interface or user terminal, which is omitted in the drawing. For example, the control unit 140 can adjust the current flowing to each of the electrode modules 111 to 117 in a range of 0.1 mA to 5 mA in response to a user input. The control unit 140 can automatically adjust the current intensity applied to each of the electrode modules 111 to 117 according to a treatment mode according to a preset program.

The non-invasive stimulation device 100 of the present invention can further include a communication module 150. The communication module 150 can perform standard short-distance communication such as WiFi or Bluetooth. The control unit 140 can transmit the subject's biometric information and information on the operation of the non-invasive stimulation device 100 to the subject's terminal (e.g., a smartphone, a computer, etc.) through the communication module 150. In addition, the control unit 140 can transmit information on the treatment mode or number of times using the non-invasive stimulation device 100 to the subject's terminal. In this case, the subject can receive guidance on the use of the non-invasive stimulation device 100 and can also receive maintenance for the product.

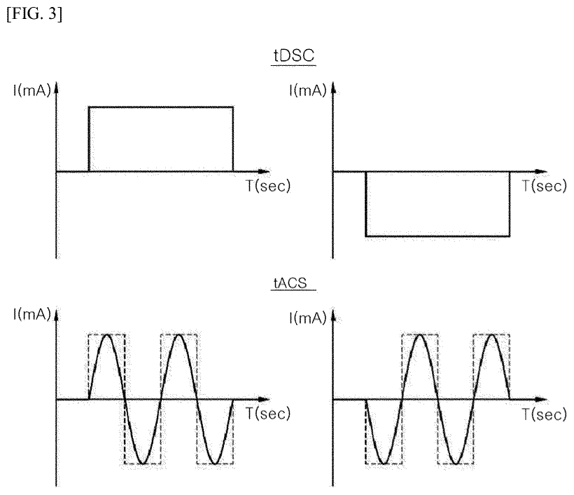

FIG. 3 is a waveform diagram schematically showing a tDCS waveform and tACS waveform in an electrical stimulation signal.

A tDCS waveform is generated as a direct current signal and applies an electrical stimulation signal of the same polarity to the electrode modules 111 to 117 for a preset treatment time. In contrast, a tACS waveform is generated as an alternating current signal and applies an electrical stimulation signal of the form of a square wave (dotted line) or sinusoidal wave (solid line) to the electrode modules 111 to 117.

tDCS regulates the voluntary neural activity of the brain through electrical stimulation of the same polarity. For example, tDCS is effective in regulating decision-making, memory, language, and sensory perception for each brain area. In contrast, tACS uses an alternating current whose polarity is periodically reversed, making it virtually impossible to control the directionality of the current (e.g., upward or downward) in the brain area. Therefore, tDCS is more widely used than tACS to prevent, treat, and manage depression, convulsive disorders, pain, intellectual disability, addiction, and mild cognitive impairment, or to improve memory and motor learning ability. However, the effect of tDCS can be reduced due to fatigue or stress of the subject. In addition, tDCS causes discomfort such as a tingling pain on the skin at the beginning of the procedure, which reduces the possibility of regular and long-term use of the non-invasive stimulation device, and even if tDCS is used, it can increase the stress of the subject. In particular, when cognitive function decline occurs, the subject's anxiety and tension increase, and the discomfort of tDCS can further increase the subject's anxiety and tension.

The non-invasive stimulation device 100 according to an embodiment of the present invention can reduce skin stimulation in the tDCS stimulation performed after tACS stimulation by adapting the subject's skin in contact with the subject's head to electrical stimulation using tACS before using tDCS. The stimulation signal of tACS can be generated as an alternating current signal of 4 to 40 Hz. Preferably, tACS (alpha) can be used. tACS (alpha) is generated as an alternating current signal of 8 to 12 Hz and has the effect of relieving the tension of the subject. Therefore, if tDCS stimulation is performed after tACS stimulation, the treatment effect is further increased.

The control unit 140 can be implemented as a program (or application) including an executable algorithm that can be executed on a computer. The program can be stored and provided in a non-transitory computer readable medium. The non-transitory computer readable medium means a medium that stores data semi-permanently and can be read by a device, rather than a medium that stores data for a short moment, such as a register, cache, or memory. Specifically, the various applications or programs described above can be stored and provided in a non-transitory computer readable medium, such as a CD, DVD, hard disk, Blu-ray disk, USB, memory card, or ROM.

The non-invasive stimulation device of the present invention can simultaneously apply electrical stimulation to multiple stimulation sites based on the diagnosis results of the patient by arranging the plurality of electrode modules. In particular, the non-invasive stimulation device of the present invention can directly and simultaneously stimulate multiple sites with abnormal brain functions, thereby improving symptom relief and treatment effects.

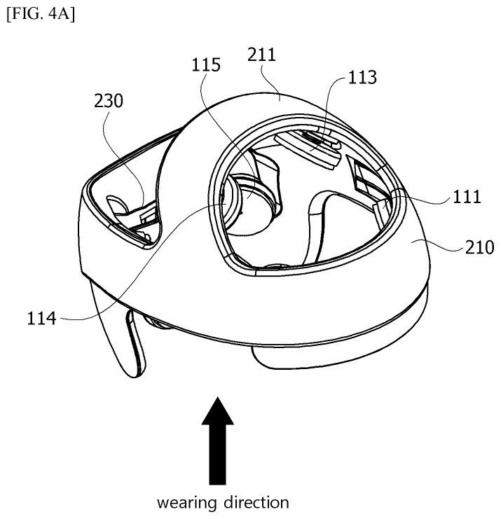

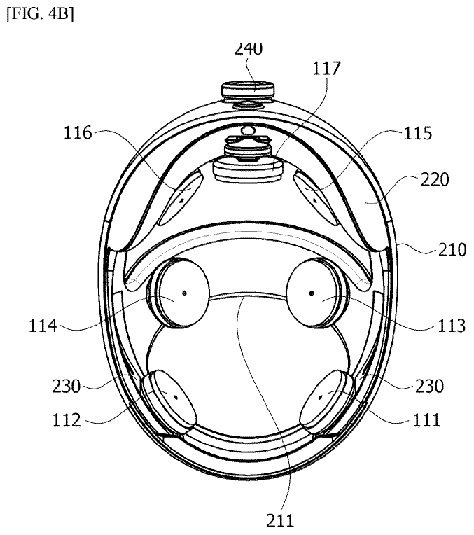

The non-invasive stimulation device according to an embodiment of the present invention can be implemented in the form of a helmet as illustrated in FIGS. 4A and 4B. FIG. 4A is a perspective view of the helmet body viewed from the upper left, and FIG. 4B is a bottom view illustrating the inside of the helmet body in a wearing direction.

Referring to FIGS. 4A and 4B, the non-invasive stimulation device 100 includes a helmet body 210, an adhesion band 230 arranged on the inner surface of the helmet body 210, at least one electrode module 111 to 117 dispersedly arranged on the adhesion band 230 and helmet body 210, and a dial 240 for tightening or loosening the adhesion band 230.

The helmet body 210 includes a bottom opening set to be larger than the head size of the subject, and is manufactured in a form that wraps from the forehead to the back of the head of the subject. One or more electrode modules 113 and 114 can be movably coupled to the upper inner surface of the helmet body 210. The upper central portion of the helmet body 210 can include a bridge part 211. Openings can be formed on both sides of the bridge part 211 in the upper portion of the helmet body 210. The electrode modules 113 and 114 can be arranged on the inner surface of the bridge part 211 facing the head of the subject. A circuit board having the power supply unit 120, control unit 140, and communication module 150 mounted thereon is built into a circuit embedded unit 220 arranged on one side of the helmet body 210, and a power button, a start key button, an LED display unit for indicating the operation status of the non-invasive stimulation device 100, a speaker, a USB port connected to an external device or power, etc., which is omitted in the drawing, can be connected to the circuit board.

The adhesion band 230 is wrapped around the inner surface of the helmet body 210 facing the head of the subject. The electrode modules 111 to 117 are flexibly distributed and installed on the adhesion band 230, the bridge part 211, and a seesaw support member, which is omitted in the drawing, so as to face each of the electrical stimulation sites illustrated in FIG. 2. The adhesion band 230 expands or contracts in diameter in conjunction with the dial 240. The adhesion band 230 can be coupled with one or more electrode modules, particularly, electrode modules facing the electrical stimulation position on the forehead of the subject. Two or more electrode modules facing the back of the head and the back of the neck of the subject can be coupled to the seesaw support member.

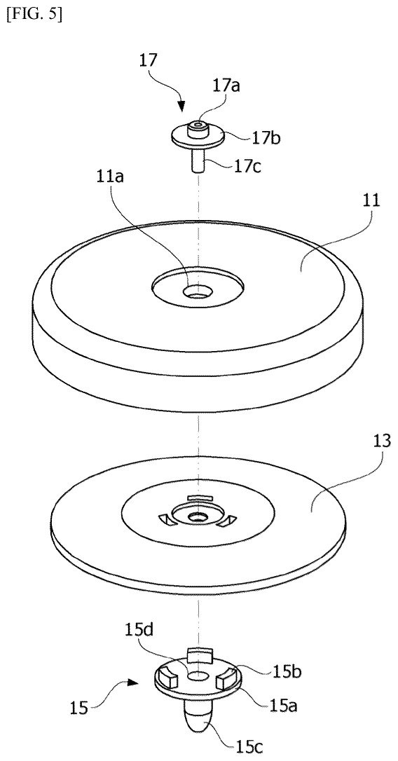

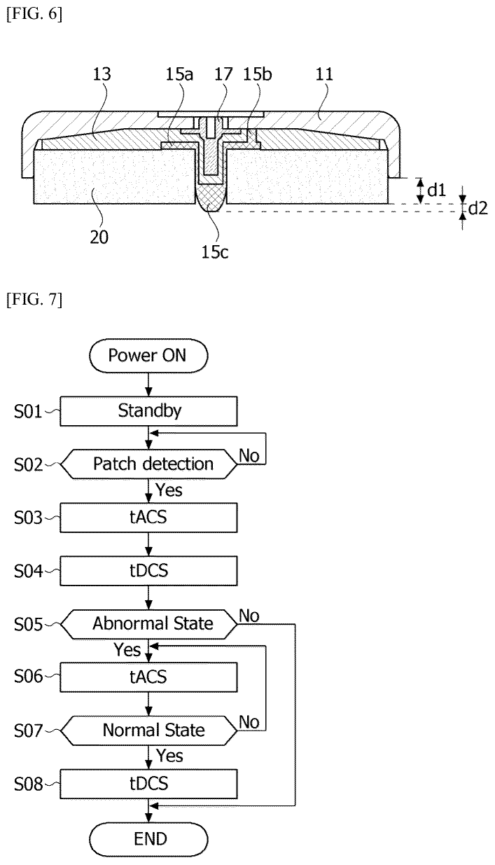

Each of the electrode modules 111 to 117 can include a non-conductive silicone holder 11, a conductive silicone pad 13, a non-conductive silicone pillar 15, a metal pin 17, and a patch 20, as illustrated in FIGS. 5 and 6.

The non-conductive silicon holder 11, conductive silicon pad 13, and non-conductive silicon pillar 15 can be formed of silicon synthetic rubber that is easy to mold. The silicon synthetic rubber has excellent heat resistance, and when combined with carbon black, silver, or a conductive material of an equivalent level, the silicon synthetic rubber has a conductivity with very low resistance.

The non-conductive silicon holder 11 is manufactured to have a container structure in which a concave internal space is surrounded by a circular belt-shaped side wall. The central portion of the non-conductive silicon holder 11 includes a hollow 11a into which a head portion 17a of a metal pin 17 is inserted.

The metal pin 17 includes the head portion 17a inserted into the hollow 11a in the concave inner surface of the non-conductive silicon holder 11, a stopper 17b protruding vertically from the outer side of the head portion 17a, and a neck portion 17c connected to the head portion 17a with a thickness thinner than the thickness of the head portion 17a. The conductive silicon pad 13 is flatly arranged on the concave inner surface of the non-conductive silicon holder 11.

The non-conductive silicon pillar 15 is bonded to the conductive silicon pad 13 at the center of the concave inner surface of the non-conductive silicon holder 11. The non-conductive silicon pillar 15 is inserted into the hollow of the patch 20 to support the patch 20.

The non-conductive silicone pillar 15 includes a wide flat portion 15a and a protrusion 15c protruding from the lower surface of the flat portion 15a. The upper surface of the flat portion 15a includes one or more small protrusions 15b. The non-conductive silicone pillar 15 includes a hollow 15d. The hollow 15d penetrates the flat portion 15a and a portion of the protrusion 15a to provide a concave space in the centers of the flat portion 15a and protrusion 15c with a depth smaller than the height of the protrusion 15c.

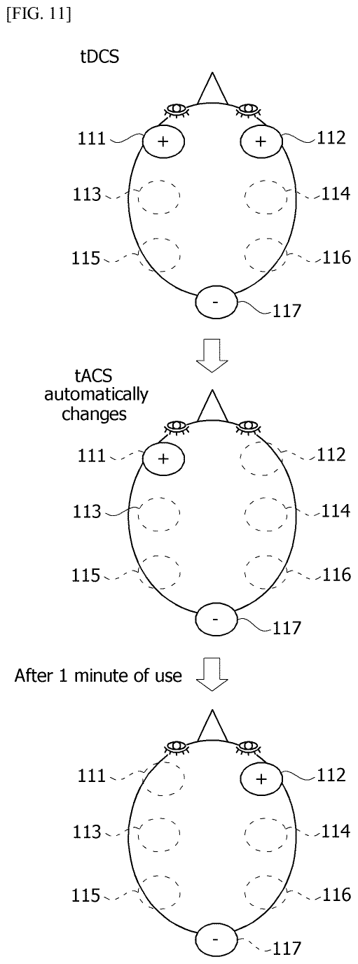

The non-conductive silicone holder 11, conductive silicone pad 13, non-conductive silicone pillar 15, and metal pin 17 can be simultaneously combined in the mold. For example, when the raw material of the conductive silicone pad 13 is injected into a mold while a separately manufactured non-conductive silicone holder 11, non-conductive silicone pillar 15, and metal pin 17 are mounted in the mold, components of the electrode module excluding the patch 20 are combined in one process as illustrated in FIG. 11.

The thin neck portion 17c of a metal pin 17 is inserted into a hollow 15d of the non-conductive silicone pillar 15. In a state in which the neck portion 17c of the metal pin 17 is inserted into the hollow 15d of the non-conductive silicone pillar 15, the conductive silicone pad 13 is filled between the metal pin 17 and the non-conductive silicone pillar 15. When the patch 20 is a wet pad, the current path and moisture penetration can spread through the conductive silicone pad 13 filled between the non-conductive silicone pillar 15 and the metal pin 17.

The protrusion 15b protruding from the flat surface 15a of the non-conductive silicone pillar 15 comes into contact with the inner surface of the non-conductive silicone holder 11, and secures a space between the inner surface of the non-conductive silicone holder 11 and the flat surface 15a of the non-conductive silicone pillar 15. The space secured by the protrusion 15b and hollow space 15d are filled with the central portion of the conductive silicone pad 13.

The patch 20 can be implemented as a wet pad or a dry pad as described above. When the patch 20 is pressed into the conductive silicone pillar 15, the protrusion 15c of the conductive silicone pillar 15 is inserted into the concave inner space of the non-conductive silicone holder 11 as illustrated in FIG. 6. The non-conductive silicone pillar 15 fixes the patch 20 within the non-conductive silicone holder 11. The thickness of the patch 20 is thicker than the side wall of the non-conductive silicone holder 11. Therefore, when the patch 20 is inserted into the non-conductive silicone holder 11, the patch 20 protrudes outward by d1 and can be closely adhered to the head of the subject.

The height of the protrusion 15c of the non-conductive silicone pillar 15 is greater than the thickness of the patch 20. Therefore, in a state in which the patch 20 is pressed into the conductive silicone pillar 15, the protrusion 15c of the non-conductive silicone pillar 15 protrudes from the patch 20 by d2 as illustrated in FIG. 6. During the electrical stimulation treatment, the central portion of the patch 20 can overheat, causing the subject to feel hot and causing a burning phenomenon in the scalp. The non-conductive silicone pillar 15 can prevent the burning phenomenon by securing a space between the patch 20 and the subject's skin in the central portion of the patch 20 and lowering the level of adhesion.

The electrode modules 111 to 117 are arranged at a position facing the electrical stimulation site inside the helmet body 210. Each of the electrode modules 117 to 117 is connected to the circuit board through wiring.

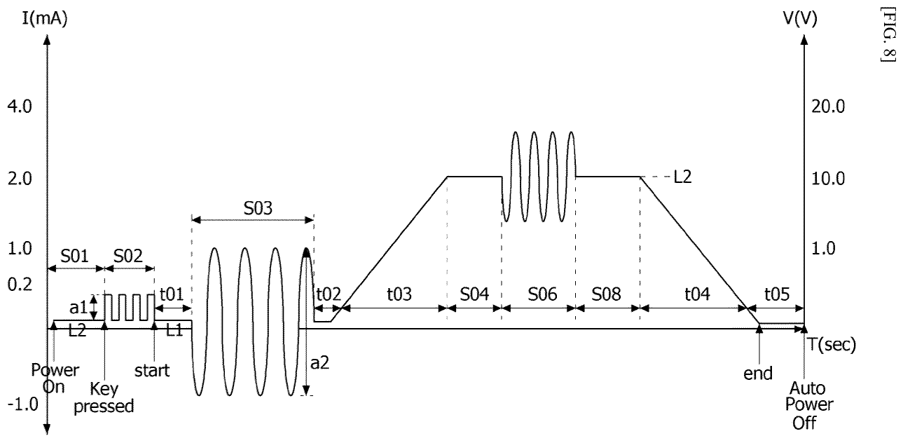

FIG. 7 is a flowchart showing a control method of a non-invasive stimulation method according to an embodiment of the present invention in time series. FIG. 8 is a waveform diagram showing an electrical stimulation signal according to an embodiment of the present invention.

Referring to FIG. 7 and FIG. 8, the non-invasive stimulation method of the present invention starts to operate when the power button is input (Power ON). When the power is input, the control unit 140 proceeds to the standby step and applies a current of a base level L1 to the electrode modules 117 for a preset initialization time (S10).

Then, when a start key button signal is input, the control unit 140 applies a patch detection signal pattern to the electrode modules 117 after a first pause time t01 to determine whether the patches of the electrode modules 111 to 117 are in close contact with the head of the subject (S02). The patch detection signal pattern is set as an alternating current signal in the form of a square wave or sine wave, and swings from the base level L1 to a predetermined first amplitude a1. When applying the patch detection signal pattern to each of the electrode modules 111 to 117, the control unit 140 can measure the voltage of each of the electrode modules 111 to 117 to determine a resistance value, and estimate a degree of adhesion of each patch. When an electrode module having a patch resistance value lower than a reference value is detected, the control unit 140 can output a warning sound or warning message to induce the subject or medical staff to correct the patch adhesion.

After all patches are adhered to the subject's head, the control unit 140 enters a first tACS step and applies a tACS stimulation signal to the selected electrode modules 111 to 117 (S03). The tACS stimulation signal is generated as an alternating current signal that repeats a negative signal lower than the base level L1 and a positive signal higher than the base level L1 during the tACS procedure and swings to a second amplitude a2 that is greater than the first amplitude a1 of the patch detection signal pattern. For example, the tACS stimulation signal is an alternating current signal of several tens of Hz, and is generated for approximately 10 minutes and can swing between-1 mA and +1 mA.

Then, the control unit 140 enters a tDCS step after a second pause time t02 and gradually increases the current intensity applied to the electrode modules 111 to 117 selected during a set ramp-up period t03. In this case, the current applied to the electrode modules 111 to 117 gradually increases from the base level L1 to a predetermined activation level L2.

The control unit 140 applies current of the activation level L2 to the electrode modules 111 to 117 for a preset tDCS treatment period after the ramp-up period t03 (S04). In a normal state where the patches are in close contact with the head of the subject, the device operates normally, and the subject's body is stable, the tDCS treatment period can be set to be longer than the tACS treatment period, for example, approximately 20 minutes.

The control unit 140 measures the voltage of the electrode modules 111 to 117 in real time while the tDCS treatment is in progress to monitor the degree of adhesion of the patches whose patch resistance value is lower than a reference value, and analyzes the biometric information from the sensor module 130 in real time to monitor the body changes of the subject. The control unit 140 temporarily suspends tDCS and automatically enters the second tACS step (S05 and S06) when the degree of adhesion of the patch is lower or the subject's physical abnormality information, such as oxygen saturation, heart rate, or stress, is out of a normal range (Abnormal state, S05). After the control unit 140 is restored to the normal state, it resumes the tDCS step and applies a stimulation signal of the activation level L2 to the electrode modules 111 to 117 (S07 and S08).

When the tDCS treatment period ends, the control unit 140 gradually lowers the current intensity applied to the electrode modules 111 to 117 to the base level L1 during a ramp-down period t04. When the tDCS treatment period ends, the output of the power unit 120 is automatically cut off after a third pause time t05 and the device stops operating.

The non-invasive stimulation device of the present invention can perform the tACS and tDCS stimulations together with a single helmet device as illustrated in FIGS. 4A and 4B. As described above, the non-invasive stimulation device of the present invention performs the tDCS treatment after the tACS treatment period, but temporarily enters the second tACS step (S06) during the tDCS treatment period when the patch resistance value increases, the device is paused, or the subject's biometric information (oxygen saturation, heart rate, stress index, etc.) exceeds a normal range. Then, when the degree of the adhesion of the patch and subject's body stabilization are confirmed, the tDCS step (S08) is resumed.

In the first tACS step (S03), the current of the electrical stimulation signal can be generated as an alternating current signal of −1 mA to 1 mA 10 Hz. When an abnormal interrupt occurs during the tDCS treatment, the current of the electrical stimulation signal generated in the automatically performed tACS step (S06) can automatically change from 0.5 mA to 1.5 mA when progressing at 1 mA per electrode, and can automatically change from 1.5 mA to 2.5 mA when progressing at 2 mA per electrode. In the second tACS step (S06), the current of the electrical stimulation signal can be generated at 8 to 12 Hz from (L2−0.5 mA) to (L2+0.5 mA) based on the activation level L2. Therefore, compared to the first tACS step (s03), the alternating current signal applied to the electrode modules 111 to 117 in the second tACS step (S06), which occurs within the tDCS treatment period, can be set to have a low amplitude and different frequencies.

The time point at which tDCS resumes after the second tACS step (S06) can be set to approximately one minute after the time point at which the subject's physical function stabilizes. If stabilization does not occur for a certain period of time, the use of the device can be automatically stopped.

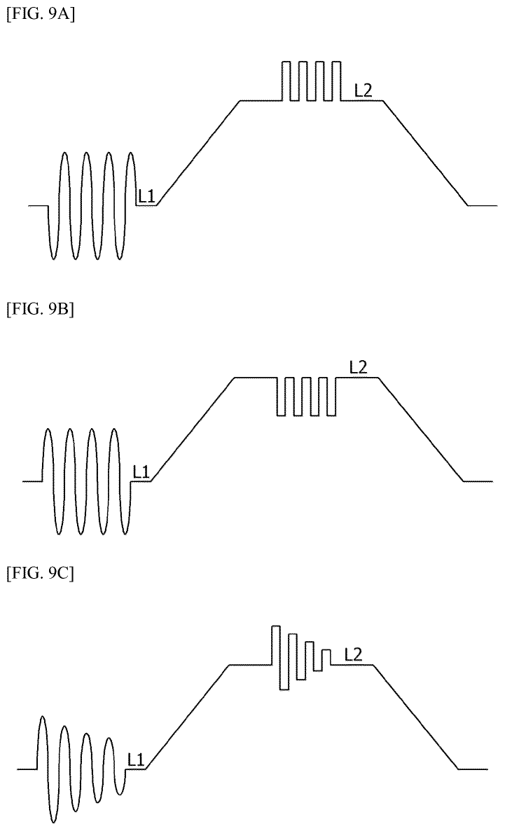

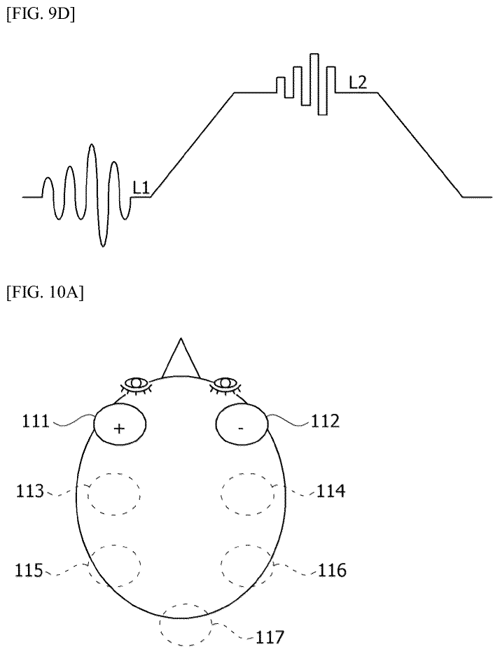

The electrical stimulation signal of the present invention is not limited to FIG. 8. For example, the electrical stimulation signal can be generated in a waveform such as FIG. 9A to FIG. 9D, and these waveforms can be combined.

The electrical stimulation signal generated in the second tACS step (S06) can be a positive alternating current waveform higher than the activation level L2 as shown in FIG. 9A, and can be a negative alternating current waveform lower than the activation level L2 as shown in FIG. 9B. In addition, in the first and second tACS steps (S03 and S06), the electrical stimulation signal can be an alternating current waveform whose amplitude gradually decreases as shown in FIG. 9C, and can be a waveform whose amplitude gradually increases and then gradually decreases as shown in FIG. 9D. Waveforms such as FIG. 9C and FIG. 9D can be more effective in reducing discomfort of the subject during the tDCS treatment period, but the waveform can be selected depending on the symptoms or condition of the subject.

The non-invasive stimulation device and method of the present invention can provide the advantages of the tACS stimulation effect while maintaining the tDCS stimulation effect by performing tACS stimulation during the tDCS treatment period. For example, a phenomenon of neural entrainment between electrical stimulation and brain waves can occur. Alpha wave entrainment can provide effects such as stable sleep or focused immersion when the alpha wave component in the brain waves becomes stronger when the brain is stimulated with the alternating current stimulation signal of alpha wave frequency. Theta wave entrainment can provide effects such as cognitive function enhancement and sleep sedation when the theta wave in the brain waves becomes stronger when the brain is stimulated with the alternating current stimulation signal of theta wave frequency. Delta wave entrainment can provide effects that can help with stable and deep sleep when the delta wave in the brain waves becomes stronger when the brain is stimulated with the alternating current stimulation signal of delta wave frequency.

The control unit 140 can switch between tACS brain stimulation and tDCS brain stimulation by controlling each of the electrode modules 111 to 117 with the negative or positive pole.

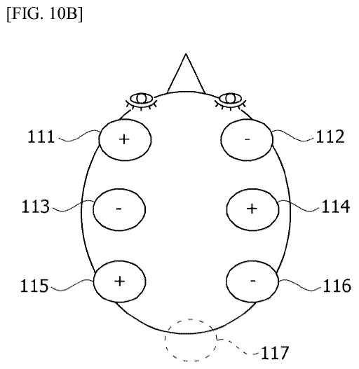

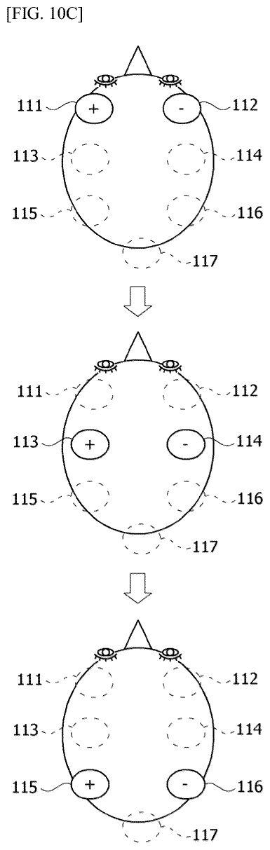

FIGS. 10A to 10C are diagrams illustrating an example of electrode polarity in tACS brain stimulation. The control unit 140 can apply an alternating current signal to a pair of electrodes selected according to the symptoms of the subject in the tACS step. For example, as shown in FIG. 10A, the first and second electrode modules 111 and 112 can be driven with opposite polarities and their polarities can be periodically reversed. In this case, other electrode modules 113 to 117 cannot be driven because no current is applied to them.

As another example, the control unit 140 can apply an alternating current signal to a plurality of electrode pairs in the tACS step. For example, as illustrated in FIG. 10B, a first electrode pair including the first and second electrode modules 111 and 112, a second electrode pair including the third and fourth electrode modules 113 and 114, and a third electrode pair including the fifth and sixth electrode modules 115 and 116 can be driven with opposite polarities, and their polarities can be periodically reversed. In this case, the seventh electrode module 117 cannot be driven.

The control unit 140 can switch the electrode pairs to which an alternating current signal is applied at the tACS step at a predetermined time cycle. For example, as illustrated in FIG. 10C, the electrodes can be driven with the alternating current signal in the order of the first electrode pair, the second electrode pair, and the third electrode pair.

FIG. 11 is a diagram illustrating an example of electrode polarity change when switching from tDCS brain stimulation to tACS brain stimulation.

Referring to FIG. 11, the control unit can apply an alternating current signal to other electrodes in the second tACS step that automatically is entered after applying an electrical stimulation signal of the same polarity to the electrodes selected in the tDCS step. In the second tACS step, the electrodes that are AC-driven can be changed at a predetermined time cycle.

Since the contents of the specification described in the problem to be solved, the means for solving the problem, and the effect described above do not specify the essential features of the claims, the scope of the claims is not limited by the matters described in the contents of the specification.

Although the embodiments of the present invention have been described in more detail with reference to the accompanying drawings, the present invention is not necessarily limited to these embodiments, and various modifications can be implemented within a scope that does not depart from the technical idea of the present invention. Therefore, the embodiments disclosed in the present invention are not intended to limit the technical idea of the present invention but to explain the present invention, and the scope of the technical idea of the present invention is not limited by these embodiments. Accordingly, it should be understood that the embodiments described above are exemplary in all respects and not restrictive. The protection scope of the present invention should be interpreted by the claims, and all technical ideas within the scope equivalent thereto should be interpreted as being included in the scope of the rights of the present invention.

### INDUSTRIAL APPLICABILITY

According to the present invention, since mild cognitive impairment can be prevented by mounting the device on equipment such as a helmet, the subject can safely stimulate the brain.

1. A non-invasive stimulation device comprising:
a helmet in which a plurality of electrodes each including a patch configured to adhere to a head of a subject are disposed;
at least one sensor module configured to sense biometric information of the subject wearing the helmet; and
a control unit configured to apply a direct current stimulation signal to at least one selected electrode in a transcranial direct current stimulation (tDCS) step, provide an alternating current stimulation signal to at least one selected electrode in a transcranial alternating current stimulation (tACS) step, and automatically switch from the tDCS step to the tACS step when an abnormal state in which a resistance value of the electrode exceeds a reference value or the biometric information exceeds a normal range is detected.

2. The non-invasive stimulation device of claim 1, wherein the control unit applies the alternating current signal that swings around a base level in a first tACS step to the electrode selected in the tDCS step,
increases an intensity of a current applied to the electrode selected in the tACS step to an activation level higher than the base level during a ramp-up period after the first tACS step,
enters a second tACS step and applies the alternating current signal that swings around the activation level to the electrode selected in the tACS step when the abnormal state is detected, and
resumes the tDCS step after a predetermined period of time when a normal state in which the resistance value of the electrode is lower than or equal to the reference value and the biometric information changes within a normal range is detected, and then gradually lowers the intensity of the current applied to the electrode selected in the tACS step from the activation level to the base level during a ramp-down period.

3. The non-invasive stimulation device of claim 2, wherein the control unit applies a current of the base level to the electrodes for a predetermined standby time immediately after a power of the non-invasive stimulation device is turned on,
applies a patch detection signal pattern in a form of the alternating current signal to the electrodes when the start key input is received, measures resistances of the electrodes, compares the resistance value of each of the electrodes with the reference value, and determines a degree of adhesion of the patch, and
enters the first tACS step after the patch detection signal pattern.

4. The non-invasive stimulation device of claim 3, wherein an amplitude of the alternating current signal generated in the first tACS step is greater than an amplitude of the patch detection signal pattern.

5. The non-invasive stimulation device of claim 2, wherein an amplitude of the alternating current signal generated in the second tACS step is lower than an amplitude of the alternating current signal generated in the first tACS step.

6. The non-invasive stimulation device of claim 2, wherein a frequency of the alternating current signal generated in the second tACS step is different from a frequency of the alternating current signal generated in the first tACS step.

7. The non-invasive stimulation device of claim 2, wherein the alternating current signal generated in at least one of the first tACS step and the second tACS step includes a section in which an amplitude changes.

8. A non-invasive stimulation method which uses a helmet in which a plurality of electrodes each including a patch configured to adhere to a head of a subject are disposed, at least one sensor module configured to sense biometric information of the subject wearing the helmet, and a control unit configured to apply a direct current stimulation signal to at least one selected electrode in a transcranial direct current stimulation (tDCS) step, and provide an alternating current stimulation signal to at least one selected electrode in a transcranial alternating current stimulation (tACS) step, wherein the method comprises the steps of:
measuring a resistance value of the electrodes and comparing the resistance value with a reference value;
receiving an output signal of the sensor module and comparing the biometric information with a normal range; and
automatically switching from the tDCS step to the tACS step when an abnormal state in which the resistance value exceeds the reference value or the biometric information exceeds the normal range is detected.

9. The non-invasive stimulation method of claim 8, further comprising the steps of:
applying the alternating current signal that swings around a base level in a first tACS step to the electrode selected in the tDCS step;
increasing an intensity of a current applied to the electrode selected in the tACS step to an activation level higher than the base level during a ramp-up period after the first tACS step;
entering a second tACS step and applying the alternating current signal that swings around the activation level to the electrode selected in the tACS step when the abnormal state is detected;
resuming the tDCS step after a predetermined period of time when a normal state in which the resistance value of the electrode is lower than or equal to the reference value and the biometric information changes within a normal range is detected; and
gradually lowering the intensity of the current applied to the electrode selected in the tACS step from the activation level to the base level during a ramp-down period after the tDCS step is resumed.

10. The non-invasive stimulation method of claim 9, further comprising the steps of:
applying a current of the base level to the electrodes for a predetermined standby time immediately after a power of the non-invasive stimulation device is turned on,
applying a patch detection signal pattern in a form of the alternating current signal to the electrodes, measuring resistances of the electrodes, comparing the resistance value of each of the electrodes with the reference value, and determining a degree of adhesion of the patch; and
entering the first tACS step after the patch detection signal pattern.
